(19) United States
(12) Patent Application Publication
(10) Pub. No.: US 2006/0099193 A1
(43) Pub. Date: May 11, 2006

(54) Methods and materials for suppressing pain

(76) Inventors: Ikuko Sugaya, Winter Park, FL (US); Kiminobu Sugaya, Winter Park, FL (US)

Correspondence Address: BEUSSE BROWNLEE WOLTER MORA & MAIRE, 390 N. ORANGE AVENUE, SUITE 2500, ORLANDO, FL, 32801, US

(21) Appl. No.: 11/258,392

(22) Filed: Oct. 24, 2005

Related U.S. Application Data
(60) Provisional application No. 60/621,903, filed on Oct. 22, 2004.

(51) Int. Cl. A61K 48/00; C12N 5/08

(52) U.S. Cl. 424/93.21; 435/368

(57) ABSTRACT

Disclosed herein are methods and materials for suppressing pain that involve the production and implantation of stem cells genetically engineered to express pain suppression proteins. Specifically disclosed is a method of implanting mesenchymal stem cells engineered to express preproenkephalin.

### CROSS REFERENCE TO RELATED APPLICATION

This application claims the benefit of U.S. Provisional Patent Application Ser. No. 60/621,903 filed Oct. 22, 2004, incorporated herein by reference

### BACKGROUND OF THE INVENTION

The spinal administration of opiate analgesics is known to relieve pain by regulating nociceptive transmission while bypassing their harmful or unpleasant side effects. However long term infusion of endogenous substance into the dorsal horn of the spinal cord, may involve some complications, including infection. Thus, alternative administration methods are desirable for chronic pain treatment. Preproenkephalin (PPE), a precursor protein for endogenous opiate, has been a good target for a gene therapy to control pain. Wilson et al. introduced PPE gene to sensory neurons by herpes-mediated gene delivery system and successfully modulate certain types of nociception reactions in a model animal (8,15,23). However effective production and release of endogenous opiate may not be achieved and dissemination of the virus could be a problem. Other possible approach is a transplantation of cells or tissue producing opioid peptide (11). Implantation of chromaffin cells is reported to produce long-term analgesic effect and promising in treating terminal cancer patients (1,9,12) . However, these cells were originated from other subjects and the patients received the transplantation need to be immunosuppressed for extended period. Furthermore it requires several donor's tissue (the medulla of adrenal grand) to treat one patient, resulting that shortage of transplantable material may occur. Recently, existences of adult stem cells, which may be expanded in a culture and differentiated into many different types of cells , attract the attention of researchers (16, 20). Adult born marrow contains not only hematopoietic stem cells involve in hematopoiesis but also mesenchymal stem cells (MeSCs) to produce connecting tissue. Since MeSCs are rather easy isolate and grow in the culture, their possibility as the vehicle for ex vivo gene therapy has been considered (5). We have previously reported human MeSCs (HMeSCs) injected into the lateral ventricle of rodents migrated into the host brain and survived a long period (17). Thus, a need exists for a pain therapy that does not suffer from the drawbacks of implantation of allogenic chromaffin cells.

### BRIEF DESCRIPTION OF THE DRAWINGS

FIG. 1 shows an image of a gel that demonstrates the presence of polynucleotides pertaining to GFP and hppe genes. Lane a: GFP and hppe genes transfcted hMSCshows EGFP gene expression (318 bp and hppe gene expressed at 425 bp; lane b:, hppe gene transfected hMeSC expressed at 425 bp lane c: 100 bp ladder.

FIG. 2 shows an image demonstrating GFP expression in HNSC after transfection with a mammalian expression vector containing GFP and hPPEP genes (FIG. 2A) and GFP expression of HMeSC after transfection with a mammalian expression vector containing GFP and hPPEP genes.

FIG. 3 is an image showing in lanes a and b: met-enkephalin expression from HNSC transfected with hppe gene; in lane c: no met-enkephalin expression showed from naive HNSC; in lanes d and e: met-enkephalin expressio from HMeSC transfected with hppe gene; and in lane f: met-enkephalin expression from naive HMeSC.

FIG. 4 is an image showing Met-enkephalin expression from HMeSCs before and after adipogenesis. lane a and b relate to met-enkephalin expression from HMeSCs lane c and d: met-enkephalin expression from adipogenic HMeSCs.

FIG. 5 shows a graph demonstrating expression of Met-enkephalin in transfected and non-transfected cells.

FIG. 6 shows a sequence for the human preproenkephalin gene.

### DETAIL DESCRIPTION

In reviewing the detailed disclosure which follows, and the specification more generally, it should be borne in mind that all patents, patent applications, patent publications, technical publications, scientific publications, and other references referenced herein are hereby incorporated by reference in this application, in their entirety to the extent not inconsistent with the teachings herein.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

Stem cells are modified to express pain suppressing proteins. In one embodiment, human mesenchymal stem cells are modified by introduction of a polynucleotide encoding a preproenkephalin protein that is expressed. The stem cells are implanted into a subject suffering from chronic pain, or about to suffer from pain, in such a manner that the pain suppressing proteins, such as, but not limited to, Met-enkephalin are delivered to the central nervous system and/or peripheral nervous system.

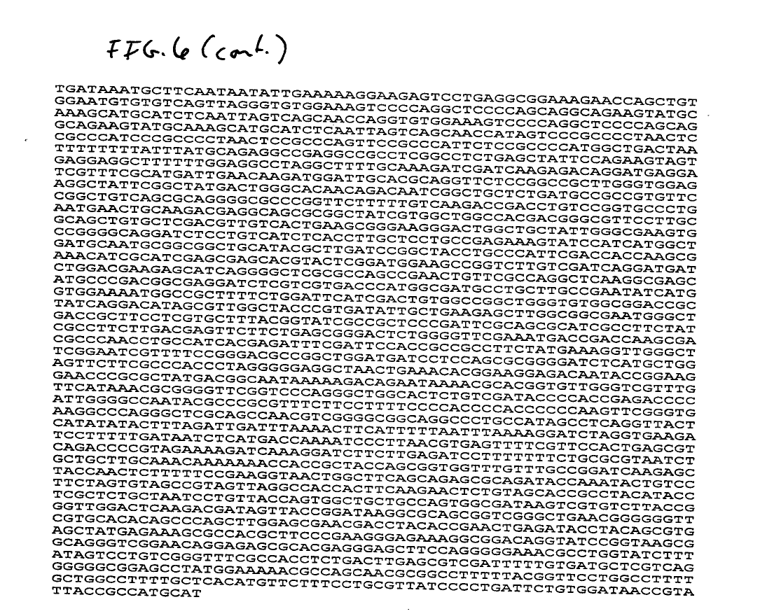

According to another embodiment, the subject invention pertains to a method of relieving pain in a subject in need thereof comprising obtaining mesenchymal stem cells from said subject, introducing into said stem cells a polynucleotide that hybridizes to the complement of the sequence in FIG. 6 and whose expression produces a gene product that suppresses pain, thereby producing modified stem cells; and implanting into said subject said modified stem cells at a location wherein said gene product that suppresses pain is delivered to the central nervous system or peripheral nervous system. Those skilled in the art will appreciate that transplantation may occur at different sites in the subject. See Cell Transplant. Sepember-October 2000; 9(5):637-56 and [1, 9, and 12] incorporated by reference. In a typical embodiment, cells are implanted at the spinal cord of the subject.

In the context of the present application, a polynucleotide sequence is “homologous” with the sequence according to the invention if at least 70%, preferably at least 80%, most preferably at least 90% of its base composition and base sequence corresponds to the sequence according to the invention. According to the invention, a “homologous protein” is to be understood to comprise proteins which contain an amino acid sequence at least 70% of which, preferably at least 80% of which, most preferably at least 90% of which, corresponds to the human Met-enkephalin protein; wherein corresponds is to be understood to mean that the corresponding amino acids are either identical or are mutually homologous amino acids. The expression “homologous amino acids” denotes those which have corresponding properties, particularly with regard to their charge, hydrophobic character, steric properties, etc. Thus, in one embodiment the protein may be from 70% up to less than 100% homologous to preproenkephalin.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The term “isolated” means separated from its natural environment.

The term “polynucleotide(s)” refers in general to polyribonucleotides and polydeoxyribonucleotides, and can denote an unmodified RNA or DNA or a modified RNA or DNA.

The term “polypeptide(s)” is to be understood to mean peptides or proteins which contain two or more amino acids which are bound via peptide bonds.

The polypeptides for use in accord with the teachings herein include polypeptides corresponding to preproenkephalin, and also includes those, at least 70% of which, preferably at least 80% of which, are homologous with the polypeptide corresponding to preproenkephalin, and most preferably those which exhibit a homology of least 90% to 95% with the polypeptide corresponding to prepoenkephalin and which act to suppress pain. Thus, the polypeptides may have a homology of from 70% to up to 100% with respect to preproenkephalin.

As used herein, a “polypeptide sequence exhibiting dedifferentiating influence” is a polypeptide whose presence in the cell causes an increase in potency, or transformation from a less developmentally potent cell to a more developmentally potent cell. Examples of such polypeptide sequences include the expression products of the preproenkaphalin gene (FIG. 6), and polynucleotide sequences that hybridize to the complement of the sequence in FIG. 6, as well as expression products of the polynucleotide sequences discussed in [20].

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tm can be approximated from the equation of Meinkoth and Wahl, Anal. Biochem., 138:267-284 (1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Current Protocols in Molecular Biology, Chapter 2, Ausubel, et al., Eds., Greene Publishing and Wiley-Interscience, New York (2000).

Accordingly, polynucleotide sequences that hybridize to the complement of the sequence in FIG. 6 are contemplated for use in dedifferentiating cells as taught herein.

U.S. Patent Application Nos. 2003/0,219,898, 2003/0,148,513, and 2003/0,139,410 are incorporated by reference to the extent they are not inconsistent with the teachings herein. These first two of these patent applications describe multiple uses of increased potency cells obtained from the taught methods, and in particular, the implantation of stem cells for different therapeutic treatments of neurological trauma and degenerative conditions.

### EXAMPLE 1

### Production of Preproenkephalin Producing Stem Cells

Cell Cultures

Human neural stem cells (HNSCs) were purchased from Cambrex (Walkersville, Md.) and cloned by its multipotency. HNSCs have been cultured at a density of 50 spheres in 75 cm2 culture flasks (Corning, Cambridge, Mass.) in 20 ml of a serum-free supplemented growth medium consisting of DMEM/F12, antibiotic-antimycotic mixture (Invitrogen, Carlsbad, Calif.), human recombinant FGF-2 and EGF (20 ng/ml each, R&D Systems, Minneapolis, Minn.), and heparin (5 μg/ml, Sigma, St. Louis, Mo. ) at 37° C. in a 5% CO2 humidified incubation chamber (Fisher, Pittsburgh, Pa.). Human mesenchymal stem cells (HMeSCs) were purchased from Cambrex, which were negative for CD11b, CD33, CD34 and CD133 antigens. HMeSCs were cultured in 20 ml of serum-supplemented growth medium consisting of Dulbecco's Modified Eagle Medium (Invitrogen), antibiotic-antimycotic mixture (Invitrogen) and FBS (Stem Cell Technologies, Vancouver, BC). The cells were incubated at 37° C. in a 5% CO2 humidified incubation chamber (Fisher). The cells were fed by replacing half the culture media twice per week. Adipogenic differenciation of HMeSCs were performed by using BulletKit (Cambrex, Walkersville, Md.) by following the manufacture's protocol.

Transfection of Human Preproenkephalin (hPPE)

The hPPE cDNA (pGEMhPPE) was a gift from Dr. S. P. Wilson (University of South Carolina, School of Medicine, Department of Pharmacology, Physiology and Neuroscience). The hPPE cDNA was dissected out form pGEMhPPE by digestion with EcoRI and HindIII. The hPPE cDNA was purified on a gel (960 bp), then, ligated into EcoRI and HindIII site of pcDNA3.1(−)/Zeo (Invitrogen), human cytomegalovirus (CMV) promoter based constitutive expression vectors (pcDNA3.1(−)/zeo/hPPE). To produce enhanced green fluorescent protein (EGFP) fusion hPPE, pcDNA3.1(−)/zeo/hPPE was digested by XhoI and HindIII and resulting fragment (993 bp) was cloned into the pEGFP (BD Bioscience, Palo Alto, Calif.) XhoI and HindIII site (pEGFP/ hPPE). All the clones were confirmed by DNA sequencing. Endotoxin free plasmids are prepared by NucleoBond Plasmid EF Kit (BD Bioscience) from bacterial cultures. Transfection of the gene to the HNSCs and HMeSCs was performed by using NeuroPORTOR (Gene Therapy Systems, Inc., San Diego, Calif.) by following the protocol provided by the manufacture. After the transfection, GFP expression was checked under the fluorescent microscope (Leica, Bannockburn, Ill.).

Gene Expression Analysis by RT-PCR

Four days after transfection, hPPE and EGFP gene expressions were analyzed by RT-PCR. RNAs were isolated form the cells using TRIzol reagent (Invitrogen) and treated with RNAase free DNAase (Promega, Madison, Wis. RT-PCR was performed using SuperScript One-Step RT-PCR with Platinum Taq (Invitrogen) using gene specific primer sets (hPPE: 5′-ACATCAACTTCCTGGCTTGCGT-3′ and 5′-GCTCACTTCTTCCTCATTATCA-3′, GFP: 5′-CAAGGACGACGGCAACTACAAGACC-3′ and 5′-CGGACTGGGTGCTCAGGTAGTGGT-3′). DNA samples resulting form RT-PCR were analyzed on 2% E-Gel (Invitrogen).

Immunoblot Assay for Metionine-Enkephalin (Met-ENK) in the Culture Media

The serum-supplemented growth medium for HMeSCs was replaced by serum-free medium (DMEM with antibiotic-antimycotic) one day before Met-ENK assay. Twenty-four hours after the medium change, medium was collected and purified with YM-30 microcon (Millipore Corp., Bedford, Mass.). Two hundreds μl of filtered sample and Met-enkephaline standards (50˜1000 ng/slot, Phoenix Pharmaceuticals, Inc., Belmont, Calif.) were applied on Hybond ECL nitrocellurose membrane (Amersham Life Science, Piscataway, N.J.) using the Slot Blot Hybridization Manifold (GENEMate, kaysville, Utah). The membranes were blocked with 3% normal donkey serum (Jackson ImmunoResearch, West Grove, Pa.) in phosphate buffer saline containing 0.5% Tween 20 (PBST) for 2 hours at room temperature. The membranes were then incubated with Met-enkephlin antibody (1:600, ImmunoStar Inc., Hudson, Wis.) for overnight at 4° C. After washing with PBST for 3 times, the membranes were incubated with anti-rabbit IgG peroxidase-linked species-specific whole donkey antibody (1:3000, Amersham Biosciences) for 90 minutes at room temperature. The membranes were washed with PBS, incubated with ECL Plus detection reagent (Amersham Biosciences) for 5 min then exposed to Hyperfilm ECL (Amersham Biosciences). The films were developed, digitally scanned into a computer and analyzed with NIH image (ImageJ, NIH). The data was normalized against cells numbers, which were counted using Bright-Line Hemacytometer (Hausser Scientific, Horsham, Pa.).

Results

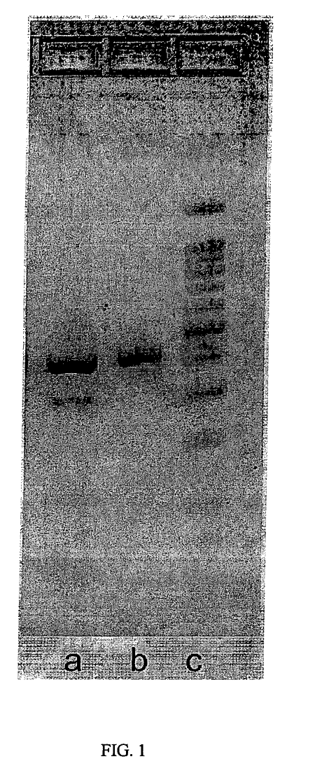

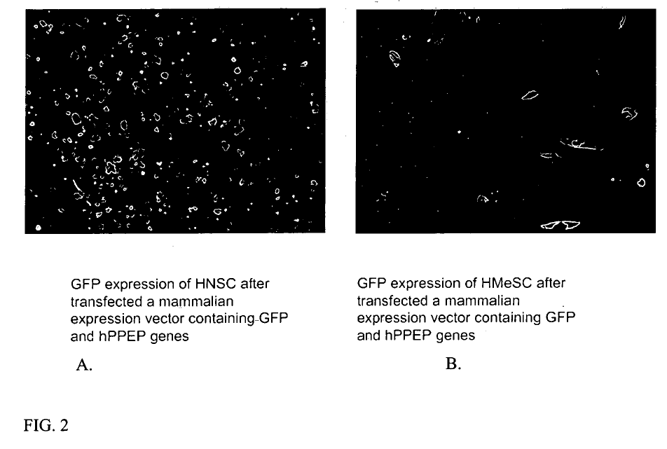

HNSCs expressed hPPE gene after transfection with pcDNA3.1(−)/zeo/hPPE, while we did not detect any significant level of hPPE gene expression in naive HNSCs. FIG. 2. Expected size for hPPE and EGFP gene fragments amplified by RT-PCR are 425 bp and 318 bp, respectively. See FIG. 1.

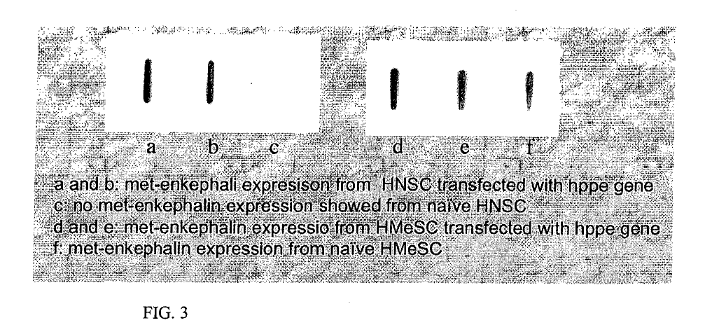

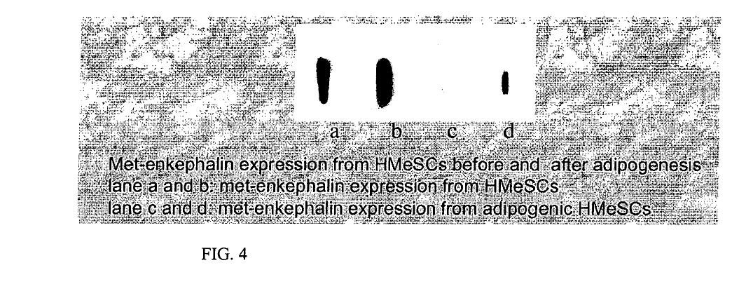

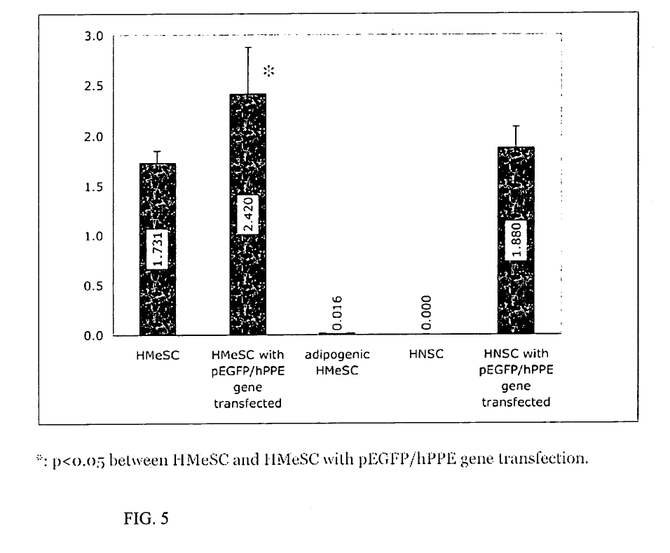

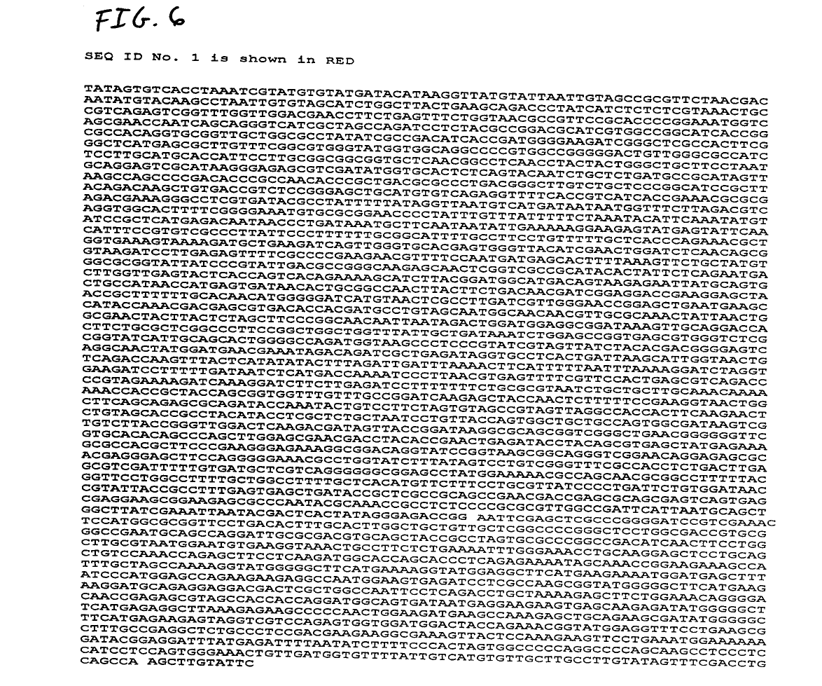

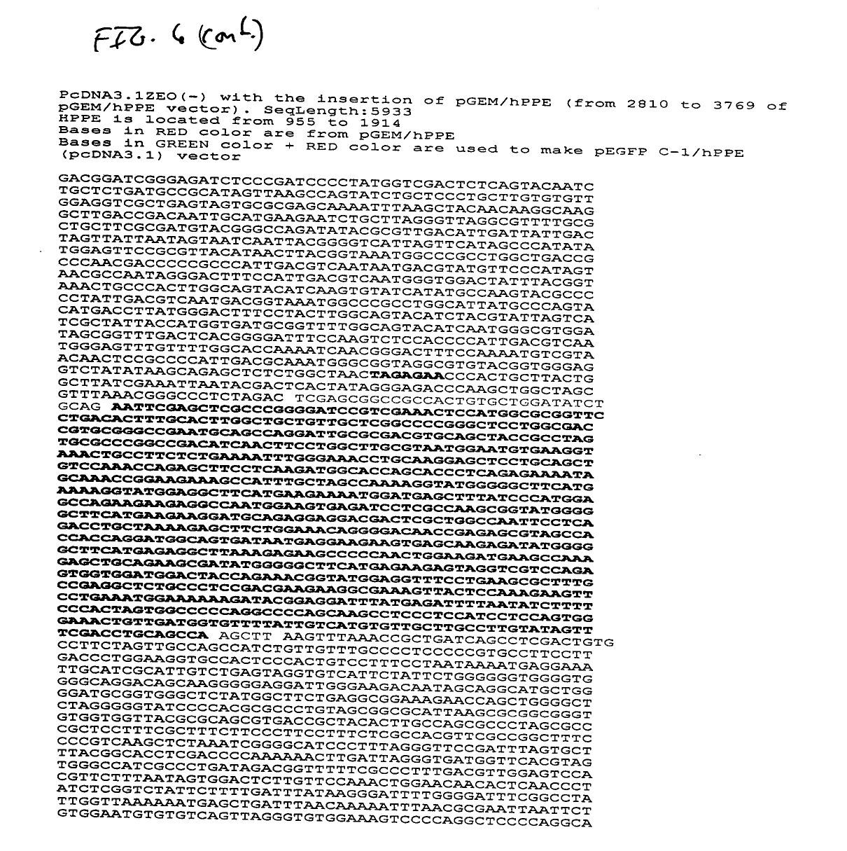

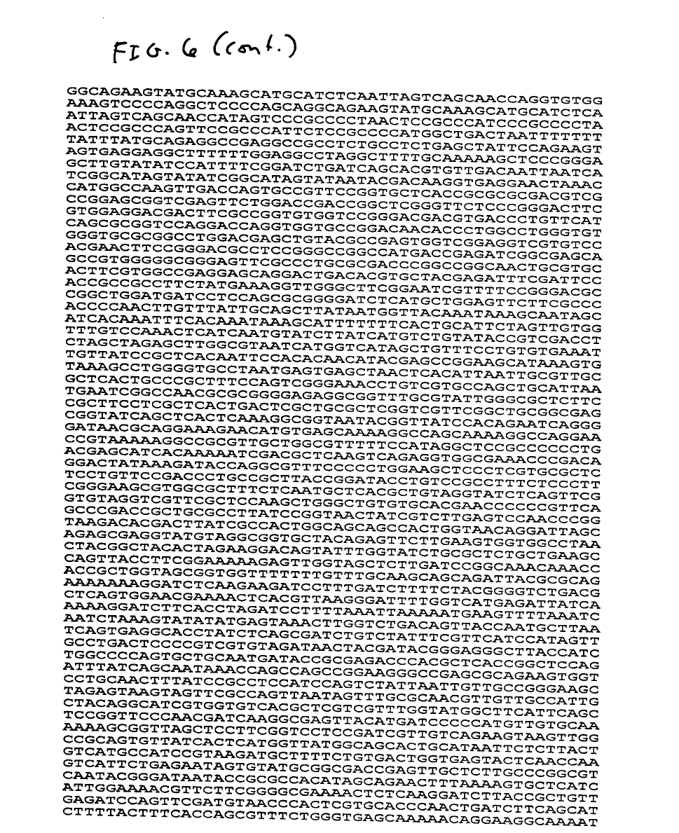

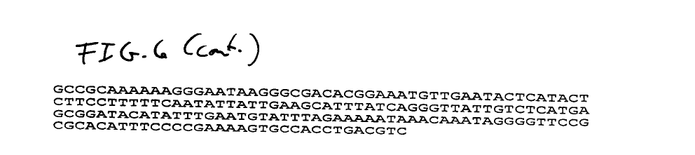

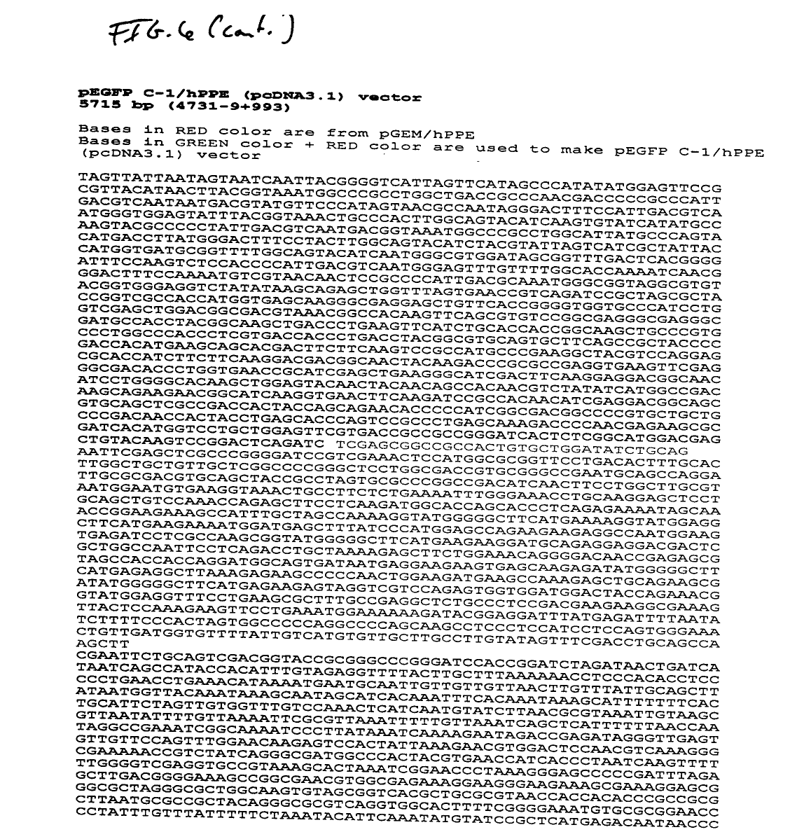

Naïve HMeSC express HPPE gene, and the hPPE gene expression became stronger after transfection with pcDNA3.1(−)/zeo/hPPE. See FIGS. 3 and 5.

After transfection with pEGFP/hPPE, HMeSC shows both hPPE and GFP gene expressions. See FIG. 2B

After transfection with pEGFP/hPPE, HNSCs produced Met-enkephalin (1.88±0.21 pg/cell) into the culture media. See FIG. 5.

Naïve HMeSC produced Met-enkephalin 1.731±0.124 pg/cell into the culture media. See FIG. 5

After transfection with pEGFP/hPPE, HMeSC produced Met-enkephalin 2.42±0.46 pg/cell into the culture media. See FIG. 5.

GFP was detected as green fluorescent under the microscope in both HNSCs and HMeSC transfected with pEGFP/hPPE. See FIG. 2.

### EXAMPLE 2

### Implantation of Mesenchymal Stem Cells in Rats Reduces Pain

We have reported that chromaffin cells, which express endogenous opioids have a potential for a treatment of chronic pain. In the current study, we combined gene and cell therapeutic approaches by engineering human neuron-committed teratocarcinoma (NT2) cells stably transfected with mammalian expression vector containing a fusion gene of human preproenkephalin and green fluorescent protein. Gene expression and production of Met-enkephalin have been tested by a RT-PCR and an immuno-assay. After stable transfection was established, the cells were terminally differentiated into neurons by retinoic acid treatment. One million cells are transplanted intrathecally between L4-L5 lumbar vertebrae level in anesthetized rats after baseline for foot withdrawal responses to noxious radiant heat mediated by Adelta and C fibers were measured. One week after the cell transplantation, foot withdrawal latencies evoked by high rate (Adelta nociceptor mediated) and low rate (C nociceptor mediated) skin heating were measured at 10 minute intervals for 1 hour. The latencies were measured once a week until the latencies back to baseline. Transplantation of the genetically engineered cells produced significant antinociceptive effect for responses to both Adelta and C thermal stimuli at similar efficacy which observed with chromaffin cell transplantation. The antinociception lasts 3 weeks. The result demonstrates that genetically engineered cells expressing preproenkephalin induced antinociception of pain sensory neurons, which may be useful for a treatment of chronic pains in humans.

### EXAMPLE 3

### Introduction into Stem Cells Neuropeptides that Alleviate Pain

Stem cells engineered according to the protocols provided in Examples 1 and 2 above are carried out to produce genetically modified cells utilizing polynucleotides that encode other neuropeptides such as proopiomelanocortin and others as described in [20].

### NUMBERED REFERENCES

1. Bes J. C., H. Tkaczuk, K. A. Czech, M. Tafani, R. Bastide, C. Caratero, G. D. Pappas, and Y. Lazorthes: One-Year Chromaffin cell allograft survival in cancer patients with chronic pain: Morphological and functional evidence, Cell Transplantation, 7(3) 227-238, 1998
2. Chang, S. C, H. L. Chuang, Y. R. Chen, J. K. Chen, H. Y. Chung, Y. L. Lu, H. Y. Lin, C. L. Tai, J. Lou: Ex vivo gene therapy in autologous bone marrow stromal stem cells for tissue-engineering maxillofacial bone regenaration, Gene Therapy 10(24) 2013-2019, 2003
3. Elcin, Y. M., A. E. Elcin, and G. D. Pappas: Functional and Morphological characteristics of Bovine Adrenal chromaffin Cells on Macroporous Poly (D,L-lactide-co-glycolide) Scaffolds, Tissue Engineering 9 (5), 1047-1056, 2003
4. Fibbe, W. E. and W. A. Noort: Mesenchymal Stem Cells and Hematopoietic Stem Cell Transplantaion, Ann. N.Y. Acad. Sci. 996, 235-244, 2003
5. Kang, W., M. A. Wilson, M. A. Bender, J. C. Glorioso, S. P. Wilson: Herpes virus-mediated preproenkephalin gene transfer to the amygdala is antinociceptive, Brain Research 792, 133-135,
6. Lazorthes Y., J. Sagen, B. Sallerin, J. Tkaczuk, H. Durplan, J. C. Sol, M. Tafani, J. C. Bes: Human chromaffin cell graft into the CSF for cancer pain managemant: a prospective phase II clinical study, Pain 87, 19-32, 2000
7. Linzhao Cheng, Holly Hammond, Zhaohui Ye, Xiangcan Zhan, Gautam Dravid: Human Adult Marrow Cells Support Prolonged Expansion of Human Embryonic Stem Cells in Culture, Stem Cells 21, 131-142, 2003
8. Liu, F., P. R. Housley, and S. P. Wilson: Initial Processing of Human Proenkephalin in Bovine Chromaffin Cells, Journal of Neurochemistry 67, 1457-1462, 1996
9. Pappas G. D., Y. Lazorthes, J. C. Bes, M. Tafini and A. P. Winnie: Relief of intractable cancer pain by human chromaffin cell transplants: Experience at two medical centers, Neurological Research, 19, 71-77, 1997
10. Pereboeva, L., S. Komarova, G. Mikheeva, V. Krasnykh, D. T. Curiel: approaches to Utilize Mesenchymal Progenitor Cells as Cellular Vehicles, Stem Cells 21, 389-404, 2003
11. Polakiewicz, R. D., O. Z. Behar, M. J. Comb and H. Rosen: Regulation of Proenkephalin Expression in Cultured Skin Mesenchymal Cells, Molecular Endocrinology 6, 399-408, 1992
12. Primeaux, S. D., M. A. Wilson, S. P. Wilson, A. N. Guth, N. B. Lelutiu, P. V. Holmes: Herpes virus-mediated preproenkephalin gene transfer in the ventral striatum mimics behavioral changes produced by olfactory bulbectomy in rats, Brain Research 988, 43-55, 2003
13. Prockop, D. J., C. A. Gregory and J. L. Spees: One strategy for cell and gene therapy: Harnessing the power of adult stem cells to repair tissue, PNAS 100 suppl. 1, 11917-11923, 2003
14. Qu, T.,
15. Rosen, H., A. Krichevsky, R. D. Polakiewicz, S. Benzakine, and Z. B. Shavit: Developmental Regulation of Proenkephalin Gene Expression in Osteoblasts, Molecular Endocrinology 9, 1621-1631, 1995
16. Rosen, H., R. D. Polakiewicz and R. Simantov: Expression of Proenkephalin a mRNA and Enkephalin-containing Peptides in Cultured Fibroblasts, Biochemical and Biophysical Research Communications 171 (2), 722-728, 1990
17. Sugiyama, O., H. Orimo, S. Suzuki, K. Yamashita, H. Ito, T. Shimada: Bone formation following transplantation of genetically modified primary bone marrow stromal cells, J. Orthop Res. 21(4), 630-637, 2003
18. Takahashi, Y and and Y. Tabata: Homogeneous Seeding of Mesenchymal Stem Cells into Nonwoven Fabric for Tissue Engineering, Tissue Engineering 9(5), 931-938, 2003
19. Weisinger, G., O. Zinder and R. Simantov: Antisense Knockout of a Neuropeptide Gene Reduces Fibroblast Proliferation, Biochemical and Biophysical Research Communications 214 (2), 530-537, 1995
20. Wilson, S. P. and D. C. Yeomans: Virally mediated Delivery of Enkephalin and Other Neuropeptide Transgenes in Experimental Pain Models, Ann. N.Y. Acad. Sci. 971, 515-521,

What is claimed is:

1. A method of relieving pain in a subject in need thereof comprising implanting into said subject a stem cell in which a polynucleotide has been introduced that expresses preproenkephalin, or a homologous protein that suppresses pain.

2. The method of claim 1, wherein said cell is a human mesenchymal stem cell or neural stem cell.

3. The method of claim 2, wherein said cell is a human mesenchymal stem cell.

4. The method of claim 1, wherein said composition is implanted into said subject such that the gene product of said it delivers said proenkephalin, or said homologous protein to the peripheral or central nervous system.

5. The method of claim 1, wherein said composition is implanted intrathecally.

6. The method of claim 1, wherein said polynucleotide hybridizes to the complement of the sequence in FIG. 6 under high stringency conditions.

7. A method of modifying a stem cell comprising introducing into said stem cell a polynucleotide that hybridizes to the complement of the sequence in FIG. 6, whose expression produces a gene product that suppresses pain.

8. A cell produced by the method of claim 6.

9. A method of relieving pain in a subject in need thereof comprising implanting into said subject a stem cell in which a polynucleotide has been introduced that expresses a gene product that suppresses pain.

10. A method of relieving pain in a subject in need thereof comprising obtaining mesenchymal stem cells from said subject, introducing into said stem cells a polynucleotide whose expression produces a gene product that suppresses pain, thereby producing modified stem cells; and implanting into said subject said modified stem cells at a location wherein said gene product that suppresses pain is delivered to the central nervous system or peripheral nervous system.

11. The method of claim 10, wherein said polynucleotide hybridizes to a complement of the sequence in FIG. 6.


SEQUENCE LISTING (8 sequences)

SEQ ID NO: 1 - DNA, 960 bp, Homo sapiens
aattcgagctcgcccggggatccgtcgaaactccatggcgcggttcctgacactttgcacttggctgctgttgctcggccccgggctcctggcgaccgtgcgggccgaatgcagccaggattgcgcgacgtgcagctaccgcctagtgcgcccggccgacatcaacttcctggcttgcgtaatggaatgtgaaggtaaactgccttctctgaaaatttgggaaacctgcaaggagctcctgcagctgtccaaaccagagcttcctcaagatggcaccagcaccctcagagaaaatagcaaaccggaagaaagccatttgctagccaaaaggtatgggggcttcatgaaaaggtatggaggcttcatgaagaaaatggatgagctttatcccatggagccagaagaagaggccaatggaagtgagatcctcgccaagcggtatgggggcttcatgaagaaggatgcagaggaggacgactcgctggccaattcctcagacctgctaaaagagcttctggaaacaggggacaaccgagagcgtagccaccaccaggatggcagtgataatgaggaagaagtgagcaagagatatgggggcttcatgagaggcttaaagagaagcccccaactggaagatgaagccaaagagctgcagaagcgatatgggggcttcatgagaagagtaggtcgtccagagtggtggatggactaccagaaacggtatggaggtttcctgaagcgctttgccgaggctctgccctccgacgaagaaggcgaaagttactccaaagaagttcctgaaatggaaaaaagatacggaggatttatgagattttaatatcttttcccactagtggcccccaggccccagcaagcctccctccatcctccagtgggaaactgttgatggtgttttattgtcatgtgttgcttgccttgtatagtttcgacctgcagcca

SEQ ID NO: 2 - DNA, 3780 bp, Artificial Sequence
  Description of Artificial Sequence Synthetic cloning vector sequence
tatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgttccgcaccccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcttatcgaaattaatacgactcactatagggagaccggaattcgagctcgcccggggatccgtcgaaactccatggcgcggttcctgacactttgcacttggctgctgttgctcggccccgggctcctggcgaccgtgcgggccgaatgcagccaggattgcgcgacgtgcagctaccgcctagtgcgcccggccgacatcaacttcctggcttgcgtaatggaatgtgaaggtaaactgccttctctgaaaatttgggaaacctgcaaggagctcctgcagctgtccaaaccagagcttcctcaagatggcaccagcaccctcagagaaaatagcaaaccggaagaaagccatttgctagccaaaaggtatgggggcttcatgaaaaggtatggaggcttcatgaagaaaatggatgagctttatcccatggagccagaagaagaggccaatggaagtgagatcctcgccaagcggtatgggggcttcatgaagaaggatgcagaggaggacgactcgctggccaattcctcagacctgctaaaagagcttctggaaacaggggacaaccgagagcgtagccaccaccaggatggcagtgataatgaggaagaagtgagcaagagatatgggggcttcatgagaggcttaaagagaagcccccaactggaagatgaagccaaagagctgcagaagcgatatgggggcttcatgagaagagtaggtcgtccagagtggtggatggactaccagaaacggtatggaggtttcctgaagcgctttgccgaggctctgccctccgacgaagaaggcgaaagttactccaaagaagttcctgaaatggaaaaaagatacggaggatttatgagattttaatatcttttcccactagtggcccccaggccccagcaagcctccctccatcctccagtgggaaactgttgatggtgttttattgtcatgtgttgcttgccttgtatagtttcgacctgcagccaagcttgtattc

SEQ ID NO: 3 - DNA, 5933 bp, Artificial Sequence
  Description of Artificial Sequence Synthetic cloning vector sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcgagctcgcccggggatccgtcgaaactccatggcgcggttcctgacactttgcacttggctgctgttgctcggccccgggctcctggcgaccgtgcgggccgaatgcagccaggattgcgcgacgtgcagctaccgcctagtgcgcccggccgacatcaacttcctggcttgcgtaatggaatgtgaaggtaaactgccttctctgaaaatttgggaaacctgcaaggagctcctgcagctgtccaaaccagagcttcctcaagatggcaccagcaccctcagagaaaatagcaaaccggaagaaagccatttgctagccaaaaggtatgggggcttcatgaaaaggtatggaggcttcatgaagaaaatggatgagctttatcccatggagccagaagaagaggccaatggaagtgagatcctcgccaagcggtatgggggcttcatgaagaaggatgcagaggaggacgactcgctggccaattcctcagacctgctaaaagagcttctggaaacaggggacaaccgagagcgtagccaccaccaggatggcagtgataatgaggaagaagtgagcaagagatatgggggcttcatgagaggcttaaagagaagcccccaactggaagatgaagccaaagagctgcagaagcgatatgggggcttcatgagaagagtaggtcgtccagagtggtggatggactaccagaaacggtatggaggtttcctgaagcgctttgccgaggctctgccctccgacgaagaaggcgaaagttactccaaagaagttcctgaaatggaaaaaagatacggaggatttatgagattttaatatcttttcccactagtggcccccaggccccagcaagcctccctccatcctccagtgggaaactgttgatggtgttttattgtcatgtgttgcttgccttgtatagtttcgacctgcagccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 5715 bp, Artificial Sequence
  Description of Artificial Sequence Synthetic cloning vector sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagcggccgccactgtgctggatatctgcagaattcgagctcgcccggggatccgtcgaaactccatggcgcggttcctgacactttgcacttggctgctgttgctcggccccgggctcctggcgaccgtgcgggccgaatgcagccaggattgcgcgacgtgcagctaccgcctagtgcgcccggccgacatcaacttcctggcttgcgtaatggaatgtgaaggtaaactgccttctctgaaaatttgggaaacctgcaaggagctcctgcagctgtccaaaccagagcttcctcaagatggcaccagcaccctcagagaaaatagcaaaccggaagaaagccatttgctagccaaaaggtatgggggcttcatgaaaaggtatggaggcttcatgaagaaaatggatgagctttatcccatggagccagaagaagaggccaatggaagtgagatcctcgccaagcggtatgggggcttcatgaagaaggatgcagaggaggacgactcgctggccaattcctcagacctgctaaaagagcttctggaaacaggggacaaccgagagcgtagccaccaccaggatggcagtgataatgaggaagaagtgagcaagagatatgggggcttcatgagaggcttaaagagaagcccccaactggaagatgaagccaaagagctgcagaagcgatatgggggcttcatgagaagagtaggtcgtccagagtggtggatggactaccagaaacggtatggaggtttcctgaagcgctttgccgaggctctgccctccgacgaagaaggcgaaagttactccaaagaagttcctgaaatggaaaaaagatacggaggatttatgagattttaatatcttttcccactagtggcccccaggccccagcaagcctccctccatcctccagtgggaaactgttgatggtgttttattgtcatgtgttgcttgccttgtatagtttcgacctgcagccaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 5 - DNA, 22 bp, Artificial Sequence
  Description of Artificial Sequence Synthetic primer
acatcaacttcctggcttgcgt

SEQ ID NO: 6 - DNA, 22 bp, Artificial Sequence
  Description of Artificial Sequence Synthetic primer
gctcacttcttcctcattatca

SEQ ID NO: 7 - DNA, 25 bp, Artificial Sequence
  Description of Artificial Sequence Synthetic primer
caaggacgacggcaactacaagacc

SEQ ID NO: 8 - DNA, 24 bp, Artificial Sequence
  Description of Artificial Sequence Synthetic primer
cggactgggtgctcaggtagtggt